(19) United States
(12) Patent Application Publication
(10) Pub. No.: US 2024/0033321 A1
(43) Pub. Date: Feb. 1, 2024

(54) PULSATIVE GNRH ADMINISTRATION FOR TREATING FOOD INTAKE RELATED DISORDERS

(71) Applicants: INSTITUT NATIONAL DE LA SANTÉ ET DE LA RECHERCHE MÉDICALE, Paris (FR); UNIVERSITÉ DE LILLE, Lille (FR); CENTRE HOSPITALIER REGIONAL UNIVERSITAIRE DE LILLE, Lille (FR); CENTRE HOSPITALIER D'ARRAS, Arras (FR); CHUV CENTRE HOSPITALIER UNIVERSITAIRE VAUDOIS, Lausanne (CH)

(72) Inventors: Vincent PREVOT, Lille cedex (FR); Nelly PITTELOUD, Lausanne (FR); Paolo GIACOBINI, Lille Cedex (FR); Valérie LEYSON, Poederlee (BE); Mauro Sérgio Batista SILVA, Lille cedex (FR); Vincent FLORENT, Lille cedex (FR); Monica IMBERNON, Lille cedex (FR)

(21) Appl. No.: 18/258,587

(22) PCT Filed: Dec. 21, 2021
(86) PCT No.: PCT/EP2021/087066

§ 371 Date: Jun. 21, 2023

(30) Foreign Application Priority Data
Dec. 22, 2020 (EP) 20306660.0

(51) Int. Cl. A61K 38/09; A61P 3/04

(52) U.S. Cl. CPC A61K 38/09; A61P 3/04

(57) ABSTRACT

The present invention relates to the pulsatile administration of the gonadotropin-releasing hormone (GnRH) for the treatment of food intake related disorder related disorder such as obesity (overeating) and anorexia (undereating). The inventors demonstrated that a population of mutant mice expressing BoNT/B in GnRH neurons (Gnrh::cre;iBot overweight), in which the GnRH release is inhibited, did not only fail to reach puberty onset and showed hypogonadotropic hypogonadism, but also developed overweight and hyperleptinemia. Unexpectedly, they found that the increase in body weight in mutant mice persisted in adulthood despite a significant decrease in food intake. Using a mouse model of obesity inventors observed an altered pattern of pulsatile LH secretion and that the frequency of LH pulses is inversely correlated to body weight and adiposity demonstrating that the heavier the animals were getting the more the GnRH/LH pulsatile pattern was altered. The present inventors further demonstrated that pulsatile GnRH treatment can allow to restore a control/lean pattern of GnRH/LH release in obese mice, and that this pulsatile administration of a “lean pattern” of native GnRH peptide in obese mice normalized their cumulative food intake to levels comparable with lean mice.

### FIELD OF THE INVENTION

The present invention pertains to novel therapeutic ways for treating food intake related disorder such as obesity (overeating) and anorexia (undereating). The present invention particularly related to the pulsatile administration of the gonadotropin-releasing hormone (GnRH) for the treatment of food intake related disorder in order to control (reducing or elevating) food intake behavior.

### BACKGROUND OF THE INVENTION

Obesity is widely recognized as a serious health problem that is increasing in prevalence across the United States and the world. According to the 1998 National Institute of Health (NIH) Clinical Guidelines on the Identification, Evaluation and Treatment of Overweight and Obesity in Adults, an estimated 97 million people in the US are classified as either overweight or obese. The medical and other costs related to obesity have risen considerably in the last two decades. In addition, many pets or companion animals, such as dogs or cats, have become obese and their owners may seek veterinary treatment to cure their obesity and associated medical problems.

Agents that have been or are currently being used for the treatment of obesity include phenylpropanolamine, dexfenfluramine, phentermine/fenfluramine, sibutramine and orlistat. Unfortunately, all of these drugs have serious adverse effects and dexfenfluramine and fenfluramine have been withdrawn because of toxicity associated with valvular heart disease in a small subset of patients.

Thus, there is a therapeutic need for safer and more effective compounds to treat obesity.

Reproductive function is tightly controlled by the hypothalamic-pituitary-gonadal (HPG)-axis, centrally orchestrated by a group of specialized neurons that synthesize and release the gonadotropin-releasing hormone (GnRH) in all vertebrates. GnRH neurons comprise a relatively small neuronal population that originate in the nose and migrate into the forebrain during embryogenesis. These neurons mostly extend fiber projections towards the median eminence (ME), where GnRH is released in a pulsatile manner into the hypophyseal portal vasculature (1). In the anterior pituitary gland, GnRH pulses drive the secretion of both gonadotropins, luteinizing hormone (LH) and follicle stimulating hormone (FSH). In turn, LH and FSH act downstream in the gonads to regulate gonadal development, gametogenesis and steroidogenesis in both sexes, and ovulation in females. Remarkably, LH secretion is pulsatile following a one to one ratio in response to GnRH pulses, which is critical for the normal functioning of the HPG axis (2). Derangement of the pulsatile release of GnRH/LH contribute to reproductive impairments and infertility (3,4). Moreover, the pulsatile administration of GnRH has been proven to successfully restore fertility in patients with hypogonadotropic hypogonadism, a rare reproductive disorder originating from GnRH deficiency or GnRH dysfunction (5), but also in patients with hypothalamic amenorrhea, including anorexia nervosa patients (Germain N. et al. Fertil Steril. 2017 February; 107(2):502-509).

Sexual development and fertility are also highly dependent upon the metabolic status, and determine the reproductive success and survival of mammalian species. Besides its key role in the regulation of reproduction, evidence is growing that GnRH is also involved in a wide-variety of non-reproductive functions, including sensing of metabolic substrates (6). GnRH neurons extend dendrites outside the blood-brain barrier (BBB) in the organum vasculosum of the lamina terminalis (OVLT), which is characterized by the presence of highly permeable fenestrated vessels (7). This access to blood-borne molecules may allow GnRH neurons to directly sense key circulating metabolic cues that usually do not cross the BBB. These findings, together with the data showing that GnRH neurons could be glucose-sensing neurons (8) suggest that GnRH neurons act as metabolic sensing neurons and could thus play a nodal role in coordinating metabolic and reproductive functions.

Both undernutrition and over nutrition are known to be associated with suppressed levels of gonadotropins and a disruption of the pulsatile pattern of LH release. These metabolic-related alterations in the reproductive axis could possibly lead to hypogonadism (9). In case of undernutrition, the presence of hypogonadism can be attributed to a deficiency in leptin, an adipokine secreted by the adipose tissue (10,11) that is known to regulate pulsatile release of neurohormones (12). In both animals and humans, sexual maturation and the age of puberty appear to be more related to body weight and adiposity, particularly in females, than to chronological age, and underweight individuals will reach sexual maturity later than normal-weight individuals (13,14). At postpubertal ages, severe undernutrition is associated with menstrual disturbances (15), a prepubertal-like appearance of the uterus and ovaries in women (16) and lower circulating levels of gonadotropins in both sexes. In case of over nutrition and obesity, hypogonadism is a frequently observed co-factor that is reversible by significant weight loss. In men, obesity characterized by low circulating levels of sex hormone-binding globulin (SHBF) and the circulating levels of free testosterone appear to be negatively correlated with increasing body mass index (BMI) and adiposity (17). Both mouse models of leptin deficiency (ob/ob) and humans with specific loss-of function mutations in the gene encoding leptin (LEP) fail to reach sexual maturity and become fertile (18,19).

Conversely, the proper functioning of the reproductive axis appears to exert a regulatory effect on the metabolic status. Hypogonadism itself is suggested to be a key risk factor for obesity and could possibly lead to increased adiposity (20,21). However, inventor's results strongly suggest that, in contrast to the current dogma, gonadal steroids do not act as linchpins in connecting fertility and metabolic disorders, but that GnRH neurons themselves, and in particular the pattern of GnRH release, may play an active role in this process.

Focusing on the importance of the Neuropilin-1 receptor (Nrp1) in GnRH neurons, a receptor that has been shown to be implicated in the migration of GnRH neurons from the nose into the forebrain during embryogenesis (Hanchate, 2012), provided the first hint for this fact. The selective knock-out of Nrp1 in GnRH neurons revealed the importance of the expression of this receptor for the development of the GnRH system, but also unexpectedly pointed to the importance of the HPG-axis in the control of pre-pubertal body weight gain. Mutant mice that carried a genetic deletion of Nrp1 in GnRH neurons (Gnrh::cre; Nrp1loxP/loxP) presented a higher number of GnRH neurons in the brain, precocious puberty and precocious body weight gain and adiposity (22). Altogether, these results suggest that the central activation of the reproductive axis, and in particular of GnRH neurons, could tightly control body weight gain during the peripubertal period.

Accordingly, it is an object of the present invention to provide alternative and improved compositions and methods for controlling food intake such as overeating and undereating.

### SUMMARY OF THE INVENTION

Accordingly, the present invention pertains to GnRH for use in the treatment for use in the prevention or the treatment of food intake related disorder in a patient in need thereof, wherein said GnRH is administered by pulsatile administration.

The present invention also relates to a compound selected in a group consisting a miR-200, a compound mimicking miR-200 and/or a miR-155, a compound mimicking miR-155 for use in the treatment of food intake related disorder in a patient in need thereof.

In a particular embodiment food intake related disorder is selected from the list consisting of 1) obesity, obesity related diseases, overweight or overeating and 2) anorexia cachexia syndrome (ACS), anorexia nervosa, underweight or under eating

### DESCRIPTION OF THE INVENTION

Method of treatment of food intake related disorder” The present invention provides methods and pharmaceutical compositions for treating food intake related disorder.

The inventors demonstrated that, a population of mutant mice expressing BoNT/B in GnRH neurons (Gnrh::cre;iBot overweight), in which the GnRH release is inhibited (FIG. 1), did not only fail to reach puberty onset (FIG. 2) and showed hypogonadotropic hypogonadism (FIG. 3), but also developed overweight and hyperleptinemia (FIG. 4). Unexpectedly, they found that the increase in body weight in mutant mice persisted in adulthood despite a significant decrease in food intake (FIG. 5), a phenomenon that is independent of the gonads. Together, these results point towards GnRH neurons establishing a link between sexual maturation and fertility, and metabolism.

Using a mouse model of obesity (FIG. 6) the present inventors observed an altered pattern of pulsatile LH secretion and that the frequency of LH pulses is inversely correlated to body weight and adiposity (FIG. 8) demonstrating that the heavier the animals were getting the more the GnRH/LH pulsatile pattern was altered.

The present inventors further demonstrated that pulsatile GnRH treatment can allow to restore a control/lean pattern of GnRH/LH release in obese mice, and that this pulsatile administration of a “lean pattern” of native GnRH peptide in obese mice normalised their cumulative food intake to levels comparable with lean mice, while it had no observable effect on the amount of food eaten by lean mice compared with their littermates that were infused with a vehicle solution (FIG. 9).

The present application thus shows that:
GnRH secretion deficiency is not only involved in puberty/fertility loss but also in metabolism disturbances and that GnRH neurone establish a link between sexual maturation and fertility, and metabolism;
A GnRH/LH pulsatile pattern is altered in a mouse model of obesity and that the frequency of LH pulses is inversely correlated to body weight and adiposity;
Pulsatile GnRH treatment allows in obese mice to normalise their cumulative food intake to levels comparable with lean mice and thus reversing metabolic impairments in this model.

Thus, without being bound by theory, the present application shows that GnRH deficiency is involved in the pathological pathways of food intake related disorder for which metabolic impairments is associated with overweight or underweight. Accordingly a pulsatile GnRH treatment in these food intake related disorders, allow to restore (reducing or elevating) the food intake behaviour. Indeed, GnRH deficiency is associated with anorexia (FIG. 5) but can result in overweight (FIG. 4). This demonstrates not only that GnRH controls food intake (and FIG. 5 shows that this effect is independent of the gonads), but also that GnRH deficiency can cause a marked uncoupling between food intake and metabolism. On the other hand, in obese mice, increased body weight and fat mass is associated with an alteration of the pattern of GnRH/LH release (FIG. 7) with a frequency decreasing with the increase in body weight (FIG. 8) and reinstating “lean pattern” of GnRH delivery in these mice is seen to normalise food intake (FIG. 9).

The present application thus demonstrates that a GnRH substitution treatment (with pulsatile administration) allows reversing metabolic impairments in food intake related disorder such as obesity (overeating) or anorexia (undereating).

Accordingly, the present invention pertains to GnRH for use in the treatment of food intake related disorder in a patient in need thereof, wherein said GnRH is administered by pulsatile administration.

The present invention particularly pertains to GnRH for use in the treatment of food intake related disorders in a patient in need thereof, wherein said GnRH is administered by pulsatile administration, said food intake related disorder being selected from the list consisting of 1) obesity, obesity related diseases, overweight or overeating and 2) anorexia cachexia syndrome (ACS), anorexia nervosa, underweight or under eating.

The present invention also pertains to a method for treating food intake related disorder in a patient in need thereof, comprising the pulsatile administration of GnRH to said patient.

GnRH is a neurohormone released in a pulsatile manner from GnRH neurons located in the hypothalamus. GnRH expression controls luteinizing hormone (LH) and follicle-stimulating hormone (FSH) secretion from the anterior pituitary. Differential GnRH pulse frequencies and amplitudes alter the secretion patterns of FSH and LH. GnRH is a decapeptide. In the context of the present invention, “GnRH” refers to the above GnRH decapeptide and to any water-soluble, ionizable form of GnRH, including free base, salts, or derivatives, homologs, or analogues thereof. In a particular embodiment, “GnRH” refers to gonadorelin, and particularly to:
GnRH hydrochloride (HCl) commercially available as FACTREL®, HRF®, and LUFORAN®; or
GnRH acetate/diacetate, commercially available as LUTRELEF®, LUTREPULSE®, KRYPTOCUR®, LHRH FERRING®, LUTAMIN®, RELISORM L®, CYSTORELIN® or RELISORM®.

Gonadorelin is a synthetic decapeptide that has the same amino acid sequence as endogenous GnRH synthesized in the human hypothalamus, and thus has the same pharmacological and toxicological profile as endogenous GnRH.

In the context of the present invention, the GnRH is administered in a “pulsatile” manner. As mentioned above, GnRH is naturally secreted with a specific pulse frequency and amplitude. Said frequency and amplitude vary according to species, genders and age. In the context of the present invention, the “pulsatile” administration reproduces the natural endogenous GnRH pulsatile peaks of a middle-age adult (i.e. between 20 and 30 years-old for humans) of the same species and gender as the patient, i.e. the GnRH frequency and amplitude observed in a middle-age adult of the same species and gender as the patient. Pulsatile GnRH administration is commonly used for the treatment of reproductive disorders such as amenorrhea and infertility resulting from hypogonadotropic hypogonadism—as e.g. Kallmann Syndrome (see Boehm et al. 2015; or e.g. Leyendecker et al. 1980; Schoemaker et al. 1981; Reid et al. 1981; Keogh et al. 1981, Hayes et al. 2013; or the ongoing clinical study referenced in the US National Library of medicine under the accession number NCT00383656). Thus, the skilled person knows the amount/frequency of administration to be used for reaching an endogenous GnRH pulsatile peak.

Typically, human endogenous GnRH pulsatile peaks vary from 25 to 600 ng/kg per pulse, with a peak every 60 to 180 minutes (see Hayes et al. 2013).

Typically, men GnRH pulsatile peaks correspond to an administration of 10 to 40 ng/kg of GnRH every 60 to 180 minutes, particularly of 20 to 30 ng/kg of GnRH every 90 to 150 minutes. A typical GnRH pulsatile peak in men is 25 ng/kg of GnRH every 120 minutes (see Boehm et al. 2015).

Typically, women GnRH pulsatile peak correspond to an administration of 50 to 100 ng/kg of GnRH every 60 to 120 minutes, particularly of 65 to 85 ng/kg of GnRH every to 110 minutes. A typical GnRH pulsatile peak in women is 75 ng/kg of GnRH every 90 minutes (i.e. 3 to 10 μg every 90 minutes—see Boehm et al. 2015 or clinical study NCT00383656).

The skilled person knows how to administer said pulsatile GnRH to the patient. GnRH is typically administered via transdermal, oral, or parenteral administration. As used herein, the term “parenteral” includes subcutaneous, intravenous, intra-arterial, intraperitoneal, intrathecal, intramuscular injection as well as infusion injections. GnRH is typically combined with pharmaceutically acceptable excipients to form a therapeutic composition suitable for transdermal or parenteral administration.

The GnRH is typically administered via transdermal delivery systems such as a pump (e.g. a portable infusion pump) that delivers GnRH boluses at specific intervals so as to reproduce the above endogenous GnRH pulsatile peaks. The LUTREPULSE® system produced and commercialized by Ferring Pharmaceuticals is an example of such a pump. Other suitable pumps are e.g. disclosed in the international patent application published under reference WO2007041386 or in U.S. Pat. Nos. 4,722,734; 5,013,293; 5,312,325; 5,336,168; and 5,372,579.

Alternatively, GnRH can be administered via grafted GnRH-producing neurons. According to this embodiment, GnRH-producing neurons are grafted to the patient so as to replace the native GnRH neurons of the patient, thereby remedying GnRH insufficiency. As demonstrated in the Example section of the present application, cell therapy based on grafting GnRH-secreting neurons allows restoring pulsatile GnRH secretion and reverses olfactory- and cognitive-associated impairments in Ts65Dn mice.

As explained in Lund et al (2013) it is possible to develop GnRH-secreting neurons from Human Pluripotent Stem Cells (hPSCs), and in particular from Human Induced Pluripotent Stem Cells (hiPSCs), e.g. hiPSCs established from healthy donor fibroblasts. The production of such GnRH-secreting neurons does not involve the destruction of human embryos.

As explained throughout the application, the present invention aims at restoring GnRH pulsatile secretion for treating food intake related disorder, particularly associated with metabolic dysfunction. GnRH expression is regulated via the action of several miRNAs. In particular, members of the miRNA-200 family and miR-155 are known to regulate Zeb 1 and Cebpb, respectively, two important repressors of GnRH promoter activators (see Messina et al (2016) as well as in the international patent application published under reference WO2017/182580). The present inventors have thus demonstrated that it is possible to restore pulsatile GnRH expression in a patient by overexpressing miRNA-200 family members (referred to as “miR-200”) and/or miR-155. The inventors have previously particularly demonstrated that hypothalamic overexpression of miR-200 resulted in a rescue of both the capacity to differentiate odors and recognize novel objects in a Down syndrome mice model (see WO2020221821). Accordingly, in a further embodiment, the present invention relates to a miR-200 and/or a miR-155 for use in the treatment of food intake related disorder in a patient in need thereof.

In a particular embodiment, the present invention pertains to a compound selected in a group consisting a miR-200, a compound mimicking miR-200 and/or a miR-155, a compound mimicking miR-155 for use in the treatment of food intake related disorder in a patient in need thereof.

In a particular embodiment food intake related disorder is selected from the list consisting of 1) obesity, obesity related diseases, overweight or overeating and 2) anorexia cachexia syndrome (ACS), anorexia nervosa, underweight or under eating

The present invention also pertains to a method for treating food intake related disorder in a patient in need thereof, comprising the administration of a therapeutically effective amount of a compound selected in a group consisting a miR-200, a compound mimicking miR-200 and/or a miR-155 a compound mimicking miR-155 to said patient.

A “therapeutically effective amount” is intended for a minimal amount of active agent (i.e. the miRNA) which is necessary to impart therapeutic benefit to a patient, i.e. in the present case for restoring GnRH pulsatile secretion in said patient.

MicroRNAs (miRs) are small, noncoding RNAs that are emerging as crucial regulators of biological processes. “MicroRNA”, “miRNA” or “miR” means a non-coding RNA of about 18 to about 25 nucleotides in length. These miRs could originate from multiple origins including: an individual gene encoding for a miRNA, from introns of protein coding gene, or from poly-cistronic transcript that often encode multiple, closely related microRNAs.

The miR-200 family contains miR-200a (human sequence accessible under reference MI0000737 in the miR database or under reference ENSG00000207607 in the Ensembl database), miR-200b (human sequence accessible under reference MI0000342 in the miR database or under reference ENSG00000207730 in the Ensembl database), miR-200c (human sequence accessible under reference MI0000650 in the miR database or under reference ENSG00000207713 in the Ensembl database), miR-141 (human sequence accessible under reference MI0000457 in the miR database or under reference ENSG00000207708 in the Ensembl database), and miR-429 (human sequence accessible under reference MI0001641 in the miR database or under reference ENSG00000198976 in the Ensembl database). By “miR-200”, it is herein referred to any miRNA of the miR200 family listed above.

MiR-155 has the sequence shown under reference MI0000681 in the miR database and under reference ENSG00000283904 in the Ensembl database.

Within the scope of the instant invention, the expression “a compound mimicking a miR-X nucleic acid” refers to the properties of said compound to exert the naturally occurring functions of said miR-X, namely bind to its site specific target nucleic acid and upregulate or downregulate the expression said target nucleic acid

All these miRNAs are known to the skilled person. They can be administered by means of any procedure known for the delivery of nucleic acids to the nucleus of cells in vivo so as to restore pulsatile GnRH expression in a patient in need thereof. In particular, miR-200 family members (or a compound mimicking miR-200) and miR-155 (or a compound mimicking miR-155) can be administered by using recombinant techniques. For example, a suitable vector may be inserted into a host cell and expressed in that cell so as to express the above miRs.

As used herein, the term “vector” refers to a nucleic acid molecule capable of transporting another nucleic acid to which it has been linked. One type of suitable vector is a viral vector (e.g., replication defective retroviruses, adenoviruses, lentiviruses and adeno-associated viruses (AAV)).

According to the present invention, pulsatile GnRH, the miR200 and/or the miR155 are administered for the treatment of food intake related disorder.

The term “food intake related disorder” or “eating disorder” refers to abnormal eating habits that negatively affect a person's physical and/or mental health such as obesity (overeating) or Anorexia (undereating). They include binge eating disorder, where people eat a large amount in a short period of time; anorexia nervosa, where people eat very little due to a fear of gaining weight and thus have a low body weight; bulimia nervosa, where people eat a lot and then try to rid themselves of the food; pica, where people eat non-food items; rumination syndrome, where people regurgitate food; avoidant/restrictive food intake disorder (ARFID), where people have a reduced or selective food intake due to psychological reasons and a group of other specified feeding or eating disorders (Arlington, VA American Psychiatric Association (2013). Diagnostic and Statistical Manual of Mental Disorders (5th ed.): pp. 329-35. Anxiety disorders, depression and substance abuse are common among people with eating disorders. These disorders include obesity, overweight or overeating.

In a particular embodiment food intake related disorder is selected from the list consisting of 1) obesity, obesity related diseases, overweight or overeating and 2) anorexia cachexia syndrome (ACS), anorexia nervosa, underweight or under eating.

The term “obesity” refers to a condition characterized by an excess of body fat. The operational definition of obesity is based on the Body Mass Index (BMI), which is calculated as body weight per height in meter squared (kg/m 2). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m².

An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m² to less than 25 kg/m².

The term “obesity-related disorders” encompasses disorders that are associated with, caused by, or result from obesity. Examples of obesity-related disorders include overeating and bulimia, diabetes, hypertension, elevated plasma insulin concentrations and insulin resistance, dyslipidemia, hyperlipidemia, breast, prostate, endometrial and colon cancer, heart disease, cardiovascular disorders, abnormal heart rhythms and arrhythmias, myocardial infarction, congestive heart failure, coronary heart disease, angina pectoris, cerebral infarction, cerebral thrombosis and transient ischemic attack. Other examples include pathological conditions showing reduced metabolic activity or a decrease in resting energy expenditure as a percentage of total fat-free mass. Further examples of obesity-related disorders include metabolic syndrome, also known as syndrome X, insulin resistance syndrome, type II diabetes, impaired fasting glucose, impaired glucose tolerance, inflammation, such as systemic inflammation of the vasculature, atherosclerosis, hypercholesterolemia, hyperuricaemia, as well as secondary outcomes of obesity such as left ventricular hypertrophy. Obesity-related disorders also include the liver abnormalities associated with obesity such as non-alcoholic fatty liver disease (NAFLD) a rising cause of cirrhosis associated to obesity and metabolic syndrome. Indeed, NAFLD can present as simple steatosis or evolve towards inflammation and steatohepatitis (NASH), with a 20% risk of cirrhosis after 20 years. “Dyslipidemia” is a major risk factor for coronary heart disease (CHD). Low plasma levels of high density lipoprotein (HDL) cholesterol with either normal or elevated levels of low density (LDL) cholesterol is a significant risk factor for developing atherosclerosis and associated coronary artery disease in humans. Dyslipidemia is often associated with and caused by obesity.

Preferred obesity-related disorders may be in particular selected from the group consisting of dyslipidemia, non-insulin-dependent diabetes mellitus, insulin resistance, metabolic syndrome, coronary heart disease, atherosclerosis and non-alcoholic fatty liver disease.

Preferably, obesity and obesity-related diseases are not of genetic origin. In particular, obesity and obesity-related diseases induced by overeating, high fat diet, and/or hyperglycaemic diet are preferably contemplated.

The term “Cachexia” refers to a state of general ill health and malnutrition. It is often associated with and induced by malignant cancer, and it is characterized by loss of appetite, loss of body mass, especially lean body mass, and muscle wasting.

Cachexia is a syndrome characterized by an involuntary loss of weight and may include one or more of progressive loss of both fat and skeletal muscle, refractoriness of weight loss to increase nutritional input, elevated resting energy expenditure (REE), decreased protein synthesis, altered carbohydrate metabolism, hyper-catabolism/increased degradation of muscle via the ATP-ubiquitin-proteasome pathway of proteolysis and of adipose tissue via lipolysis, asthenia, anemia, chronic fatigue, nausea, and loss of bone mass. Typically, at least 5% or 5 pounds of pre-illness body weight must have been lost before the patient is diagnosed with cachexia.

Of course, one or more of the above symptoms may or may not be present in a given subject depending on the underlying disease or condition associated with it and of the treatment already received by the subject for treating the underlying disease or condition. The above symptoms or physiological conditions may also be present at various degrees. Cachexia may or may not be associated with anorexia.

The term “Anorexia nervosa”, often referred to simply as “Anorexia” refers simply to a loss of appetite, whether brought on by medical or psychological factors. Anorexia is often closely associated with, and generally contributes to, the cachexia seen in patients with advanced cancers.

Anorexia is a medical term for appetite loss. Manifestations of anorexia include a decreased sense of taste and smell of food, early satiety, a decreased sense of hunger and even outright aversion of food.

Many people with anorexia see themselves as overweight even though they are, in fact, underweight. They often deny that they have a problem with low weight. They weigh themselves frequently, eat small amounts and only eat certain foods. Some exercise excessively, force themselves to vomit, or use laxatives to lose weight. Complications may include osteoporosis, infertility and heart damage, among others. Women will often stop having menstrual periods.

The cause of Anorexia is currently unknown. Cultural factors appear to play a role, with societies that value thinness having higher rates of disease (Attia E (2010). Annual Review of Medicine. 61 (1): 425-35). Anorexia often begins following a major life-change or stress-inducing event. The diagnosis requires a significantly low weight. The severity of disease is based on body mass index (BMI) in adults with mild disease having a BMI of greater than 17, moderate a BMI of 16 to 17, severe a BMI of 15 to 16, and extreme a BMI less than 15. In children a BMI for age percentile of less than the 5th percentile is often used.

Globally, anorexia is estimated to affect 2.9 million people as of 2015 (Vos T. et al (2016) Lancet. 388 (10053): 1545-1602). It is estimated to occur in 0.9% to 4.3% of women and 0.2% to 0.3% of men in Western countries at some point in their life (Smink F R, et al. (2012). Current Psychiatry Reports. 14 (4): 406-14). About 0.4% of young women are affected in a given year and it is estimated to occur ten times more commonly among women than men (Smink F R, et al. (2012). Current Psychiatry Reports. 14 (4): 406-14)). Often it begins during the teen years or young adulthood. While anorexia became more commonly diagnosed during the 20th century it is unclear if this was due to an increase in its frequency or simply better diagnosis. Eating disorders also increase a person's risk of death from a wide range of other causes, including suicide. About 5% of people with anorexia die from complications over a ten-year period, a nearly six times increased risk (Espie J. et al (2015). Adolescent Health, Medicine and Therapeutics. 6: 9-16. The term “anorexia nervosa” was first used in 1873 by William Gull to describe this condition (Gull W W (1997). Obesity Research. 5 (5): 498-502.

The terminology Anorexia-Cachexia Syndrome (ACS) is a generic term used by physician as a diagnostic of patients having either anorexia or cachexia. As used herein therefore, ACS designates anorexia or cachexia. Diseases or conditions associated with or likely to be associated with ACS, include but are not limited to, cancer, immunodeficiency disorders such as AIDS, other infectious diseases including viral, bacterial and parasitic diseases, sepsis, rheumatoid arthritis and chronic diseases of the bowel, liver, kidneys, lungs and heart including congestive heart failure and chronic organ failure. It can also manifest itself as a condition in aging or as a result of physical traumas and burn injuries.

More specifically, diseases, conditions or disorders that are typically associated with cachexia include, but are not limited to, cancer, AIDS, liver cirrhosis, diabetes mellitus, chronic renal failure, chronic obstructive pulmonary disease, chronic cardiac failure, immune system diseases (e.g., rheumatoid arthritis and systemic lupus erythematosus), tuberculosis, cystic fibrosis, gastrointestinal disorders (e.g., irritable bowel syndrome and inflammatory bowel disease), Parkinson's disease, dementia, major depression, anorexia nervosa, an aged condition and sarcopenia. More typically, the diseases, conditions or disorders that are associated with cachexia include, but are not limited to, cancer, AIDS, liver cirrhosis, chronic renal failure, chronic obstructive pulmonary disease, chronic cardiac failure, immune system diseases (e.g., rheumatoid arthritis and systemic lupus erythematosus), tuberculosis, cystic fibrosis, gastrointestinal disorders (e.g., irritable bowel syndrome and inflammatory bowel disease), Parkinson's disease, dementia, major depression, anorexia nervosa, an aged condition and sarcopenia. Cachexia is a strong independent risk factor for morbidity and mortality. For example, cancer cachexia occurs in about half of all cancer patients and is more common in patients with lung and upper gastrointestinal cancers. Cancer patients with an involuntary 5% weight loss have a shorter median survival rate than patients with stable weight. Cancer patients with weight loss can respond poorly to chemotherapy and also can require increased chemotherapy treatments. The fact that a large proportion of cancer patients have cachexia, coupled with the demonstrated relationship between cachexia and mortality, has provided impetus for the search into underlying mechanisms and therapies that might prevent or reverse cachexia and provide a model for identifying additional therapies.

An “underlying disease or condition” is a disease or condition that is associated with ACS or that is likely to be associated with ACS.

As used herein Cancer Anorexia-Cachexia-Syndrome (CACS) is intended to include any form of cancer associated with ACS or likely to be associated with ACS. Non-limiting examples of cancers that are most often associated with ACS include gastric cancer, pancreatic cancer, non-small cell lung cancer, small cell lung cancer lung cancer, prostate cancer, colon cancer, non-Hodgkin's lymphoma, sarcoma, acute non-lymphocytic leukaemia and breast cancer.

In an embodiment, a subject in need thereof is a subject diagnosed with ACS or having a disease or condition that is likely to be associated with ACS. Subjects having cancer or AIDS are examples of likely candidates.

In an embodiment, a subject in need thereof is a subject suffering from cancer. In another embodiment, the subject in need thereof is a subject suffering from cancer but which has not yet developed ACS.

In an embodiment, a subject in need thereof is a subject suffering from an immunodeficiency such as AIDS.

In a further embodiment, the subject in need thereof is a subject which has lost at least 5%, 8%, 10%, 12%, 15% or more of his/her initial weight prior to the onset of ACS.

In another embodiment, the subject in need thereof is a subject which has lost at least 5%, 8%, 10%, 12%, 15% or more of his/her weight within a six-month period.

In a further embodiment, the subject in need thereof is a subject that is desirous of increasing his/her appetite and/or weight.

In yet another embodiment, a subject in need thereof is a subject undergoing therapy for the underlying disease or condition which is associated with ACS or likely to be associated with ACS.

Thus, in one aspect of the present invention the pharmaceutical composition comprising GnRH which is administered by pulsatile administration prior to the onset of ACS as a preventive measure.

In another aspect of the present invention the pharmaceutical composition of the present invention is administered in combination with a drug or drugs used to treat the underlying disease or condition. In a further aspect, the composition of the present invention is administered once the subject has been diagnosed with ACS. In another embodiment, the composition of the present invention is administered in combination with one or more other drugs or food supplements used for the prevention and/or treatment of ACS.

A further object of the invention relates to a method for the prevention and/or the treatment of anorexia, cachexia, underweight or under eating in a mammal in need thereof in a mammal in need thereof, comprising administering by pulsatile administration the individual with a prophylactically or therapeutically effective quantity of GnRH.

Finally, the present invention also relates to a method for the prevention and/or the treatment of food intake related disorder in a subject in need thereof, comprising the pulsatile administration of GnRH to said patient

In the context of the invention, the term “treating” or “treatment” means reversing, alleviating, inhibiting the progress of, or preventing the disorder or condition to which such term applies, or one or more symptoms of such disorder or condition. In particular, the treatment of the disorder may consist in the treatment of food intake related disorder in a mammal in need thereof.

In particular, “treatment” of obesity and obesity-related disorders may refer to the administration of the compounds of the present invention to reduce or maintain the body weight of an obese subject. One outcome of treatment may be reducing the body weight of an obese subject relative to that subject's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. Another outcome of treatment may be to maintain weight loss.

In particular, “prevention” of obesity and obesity-related disorders may refer to the administration of the compounds of the present invention to reduce or maintain the body weight of a subject at risk of obesity. One outcome of prevention may be reducing the body weight of a subject at risk of obesity relative to that subject's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Another outcome of prevention may be to prolong resistance to weight gain. Another outcome of prevention may be to prevent weight regain. Moreover, if treatment is commenced in already obese subjects, such treatment may prevent the occurrence, progression or severity of obesity-related disorders.

According to the invention, the term “patient” or “subject” or “individual” to be treated is intended for a human or non-human mammal (such as a rodent (mouse, rat), a feline, a canine, a pig or a primate). In a particular embodiment, the patient is a human.

### Pharmaceutical Composition and Kits

The invention also relates to a pharmaceutical composition comprising GnRH as defined above, and optionally a pharmaceutically acceptable carrier GnRH for use in the treatment of food intake related disorder in a patient in need thereof, wherein said GnRH is administered by pulsatile administration.

In a particular embodiment food intake related disorder is selected from the list consisting of 1) obesity, obesity related diseases, overweight or overeating and 2) anorexia cachexia syndrome (ACS), anorexia nervosa, underweight or under eating.

Preferably the pharmaceutical composition also comprises a pharmaceutically acceptable carrier.

The expression “pharmaceutically acceptable carrier” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can notably be formulated for an intravenous, intramuscular, subcutaneous, inhaled/intranasal, oral and rectal administration, and the like.

The pharmaceutical compositions of the invention may contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

To prepare pharmaceutical compositions, an effective amount of the receptor agonist may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. The form is preferably sterile and is fluid to the extent that easy syringability exists. It is preferably stable under the conditions of manufacture and storage and is preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A composition of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions can be prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

The invention will be further illustrated by the following figures and examples. However, these examples and figures should not be interpreted in any way as limiting the scope of the present invention.

### FIGURES

FIG. 1. (A) Schematics of the genetic model expressing BoNT/B in GnRH neurons. (B) KCl-induced GnRH release from living hypothalamic explants containing the median eminence in control (Gnrh::cre) and mutant (Gnrh::cre;iBot) mice.

FIG. 2. (A) Cumulative percentage of control (Gnrh::cre) and mutant (Gnrh::cre;iBot) mice showing vaginal opening (left) and their corresponding bar graphs (right). (B) Cumulative percentage of control (Gnrh::cre) and mutant (Gnrh::cre;iBot) mice that undergo the first estrous and reaches puberty onset.

FIG. 3. (A) Representative examples of estrous cycle profiles in control (Gnrh::cre) and both groups of mutant (Gnrh::cre;iBot) mice. Di: diestrus, Es: estrus, Pro: proestrus. (B) Mean uterine weight (mg/g of body weight) in females subjected to a male-induced LH-surge protocol. One-way ANOVA F(2,20)=23.36, p<0.0001, overweight Gnrh::cre vs. other groups p<0.0001 (***). (C) Plasma LH levels after exposure to male odor. ND, not detected. (D) Plasma LH levels before and after GnRH injection showing that pituitary function is intact in Gnrh::cre;iBot mice. (E) Mean size of the ovaries in control (Gnrh::cre) and both groups of mutant (Gnrh::cre;iBot) mice.

FIG. 4. (A) Body weight curves during postnatal development of control (Gnrh::cre) and both groups of mutant (Gnrh::cre;iBot) mice. Repeated measures ANOVA, F(2,18)=81.12, p<0.0001. (B) Circulating plasma leptin levels in control (Gnrh::cre), both groups of mutant (Gnrh::cre;iBot) mice and ovariectomized (OVX) mutant mice.

FIG. 5. (A) Food intake of control (Gnrh::cre), ovariectomized controls (Gnrh::cre)(OVX) and both groups of mutant (Gnrh::cre;iBot) female mice. (B) Food intake of control (Gnrh::cre), orchiectomized controls (Gnrh::cre)(ORX) and both groups of mutant (Gnrh::cre;iBot) male mice.

FIG. 6. Metabolic phenotype of the mice used. (A) Body weight of lean and obese mice. Unpaired two-tailed Student's t-test, n=7 and 8. (B) Relative percentage of fat mass of lean and obese mice. Unpaired two-tailed Student's t-test, n=7 and 8.

FIG. 7. (A-B) Representative pulsatile profile of a lean (A) and obese (B) mouse. (C) Mean LH values in lean and obese mice. Unpaired two-tailed Student's t-test, n=7 and 8. (D) Mean basal LH values in lean and obese mice. Mann-Whitney U test, n=7 and 8. (E) LH integrated response. Unpaired two-tailed Student's t-test, n=7 and 8.

FIG. 8. (A) Correlation between body weight and LH pulse frequency in obese mice. Spearman correlation analysis, n=8. (B) Correlation between relative fat mass and LH pulse frequency in obese mice. Spearman correlation analysis, n=8.

FIG. 9. Cumulative food intake of lean and obese mice receiving pulsatile administration of Lutrelef or a vehicle solution. Two-way ANOVA, F(42, 140)=1.79; p=0.007 (Time×Column Factor), n=3, 4, 3, 4.

FIG. 10. Bodyweight (A), RER (B and C), and cumulative food intake (D and E) change after Lutrelef treatment in overweight Gnrh::cre;iBot female mice. Mann-Whitney U test, n=3, 2 and 3.

FIG. 11. Bodyweight (A) and cumulative food intake (B and C) change after Lutrelef treatment in an overlean Gnrh::cre;iBot male mouse. n=3, 2 and 1.

### EXAMPLE

Material and Methods

Animals

Adult lean and obese male mice were housed under specific pathogen-free conditions in a temperature-controlled room (21-22° C.) with a 12 h light/dark cycle. Animals were provided with ad libitum access to food and water. The body weight of the animals and their food intake were followed on a daily base. Animal studies were approved by the Institutional Ethics Committee for the Care and Use of Experimental Animals of the University of Lille; all experiments were performed in accordance with the guidelines for animal use specified by the European Union Council Directive of Sep. 22, 2010 (2010/63/EU).

Body Composition

Body mass composition (lean tissue mass, fat mass, and total water content) was analyzed using a body composition scanner by nuclear magnetic resonance (NMR) technology (Minispec mq series, Bruker, Billerica, MA, USA) according to manufacturer's instructions.

Pulsatile LH Measurements

Mice were habituated for at least 10 days prior to blood sampling. Blood samples (4 μL) were collected from the tail in a 6-min interval for 2 h (between 09:00 and 12:00) and diluted in 50 μL of phosphate buffered saline (PBS)-0.05% Tween20 (pH=7.4). Samples were snap frozen in dry ice and stored at −80° C. ELISA was performed according to a previously published protocol (Czieselsky et al. 2016). Briefly, high-affinity binding 96-well microplate were coated with 50 μL of capture antibody anti-bovine LH beta subunit (518B7; provided by Lilian E. Sibley, UC Davis). A standard curve was prepared using a serial dilution of LH-RP reference (provided by Albert F. Parlow; National Hormone and Pituitary Program, Torrance, California, USA). The LH assay intra-assay coefficient of variation was 7.81% and the inter-assay coefficient of variation was 9.20%.

Pump Implantation

All mice were subcutaneously implanted with programmable micro-infusion pumps (SMP-300, iPRECIO, Japan) to receive pulsatile infusions of vehicle or Lutrelef (0.25 (is delivered over 10 min, every 3 h in males and every 2 h in females), mimicking the GnRH/LH pulsatility reported in wt mice (Czieselsky et al. 2016), with a basal low dose infusion rate (0.0025 μg/10 min) the rest of the time.

Statistics

All statistical analyses were performed using Prism 8 (GraphPad software) and assessed for normality (Shapiro-Wilk test) and variance, when appropriate. Statistical differences were evaluated using an unpaired two-tailed Student's t-tests for comparison of two groups. When the criteria for normality or equal variance were not met, a Mann-Whitney U test was used for the comparison of two groups. A Pearson or Spearman correlation analysis was performed when appropriate to study correlation. For comparison of two groups a two-way analysis of variance (ANOVA) with Tukey's post hoc tests was used. The significance level was set at p<0.05. Data are presented as means±S.E.M

### Example 1

### Results

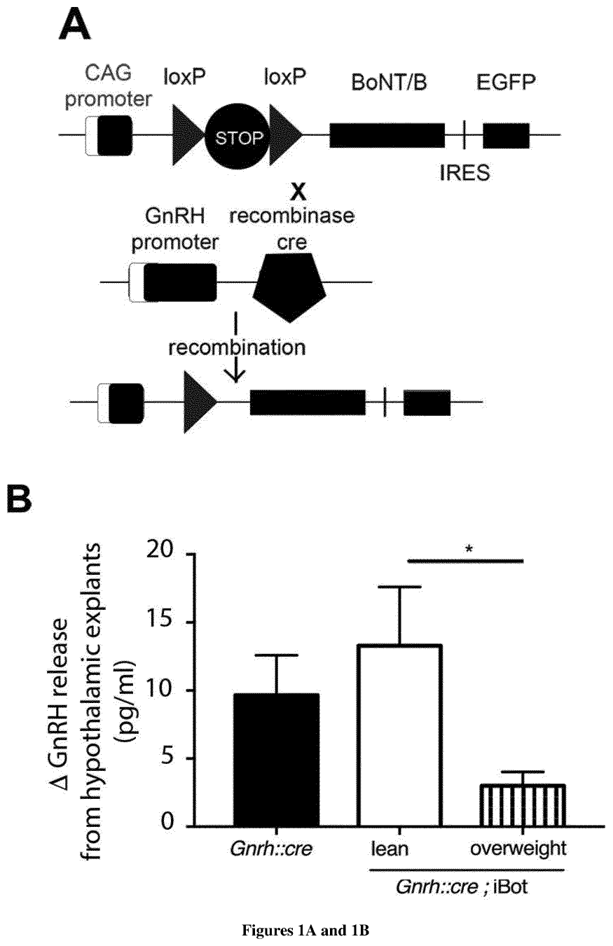

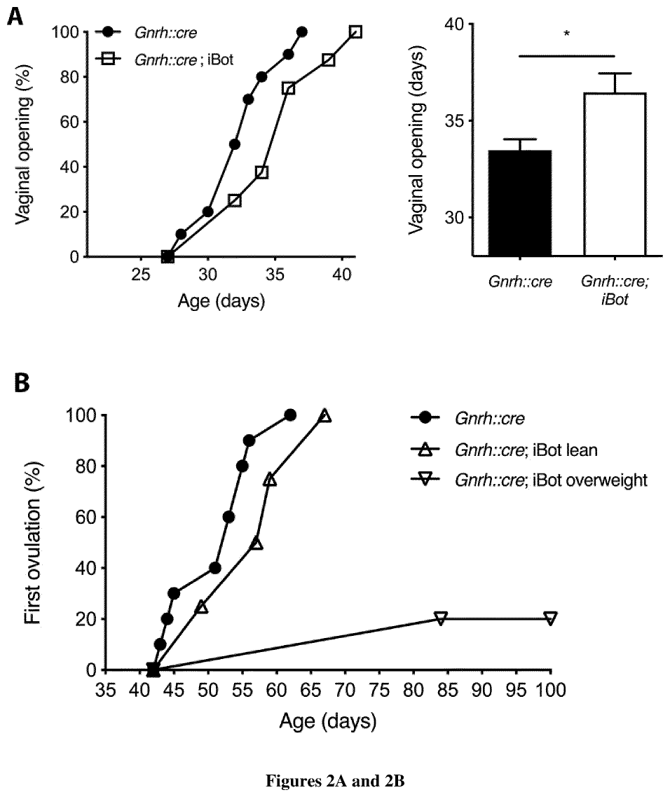

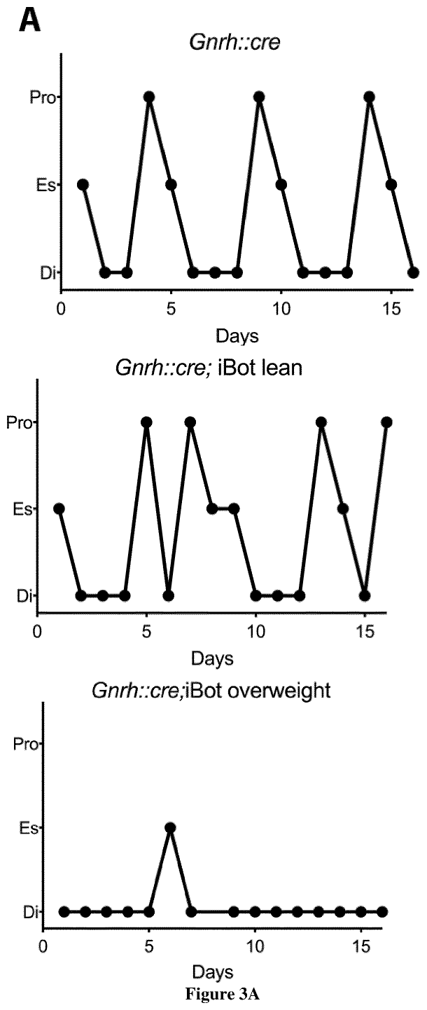

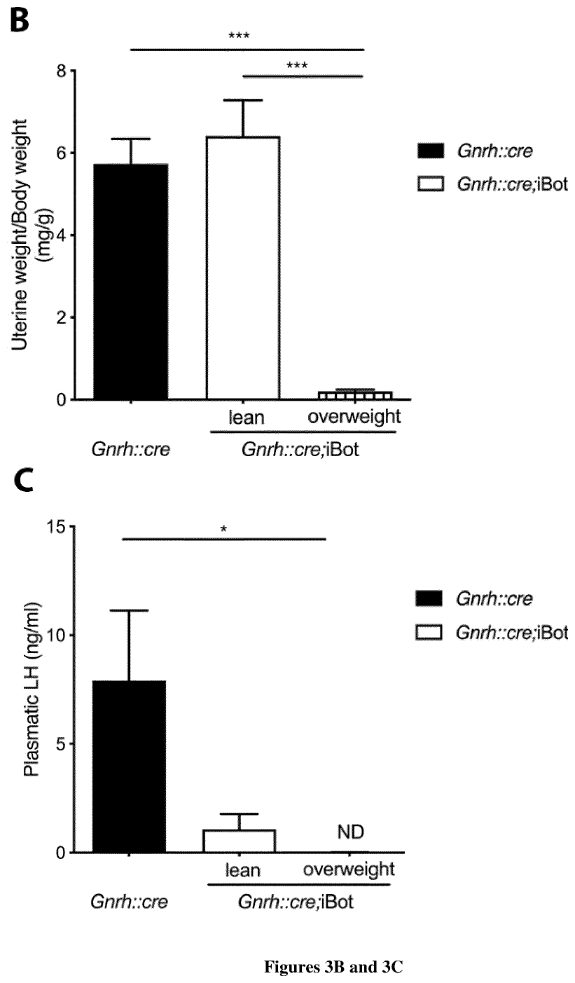

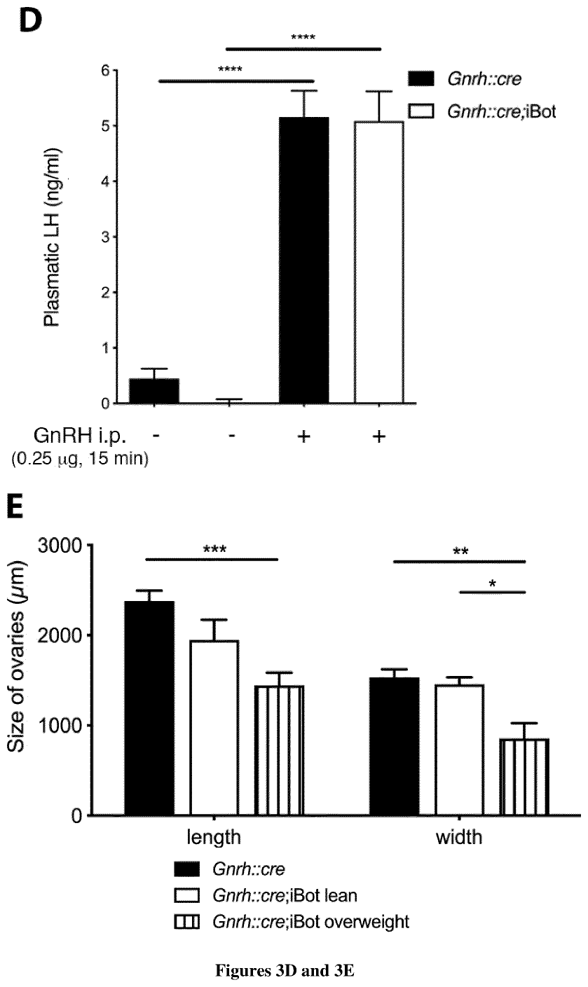

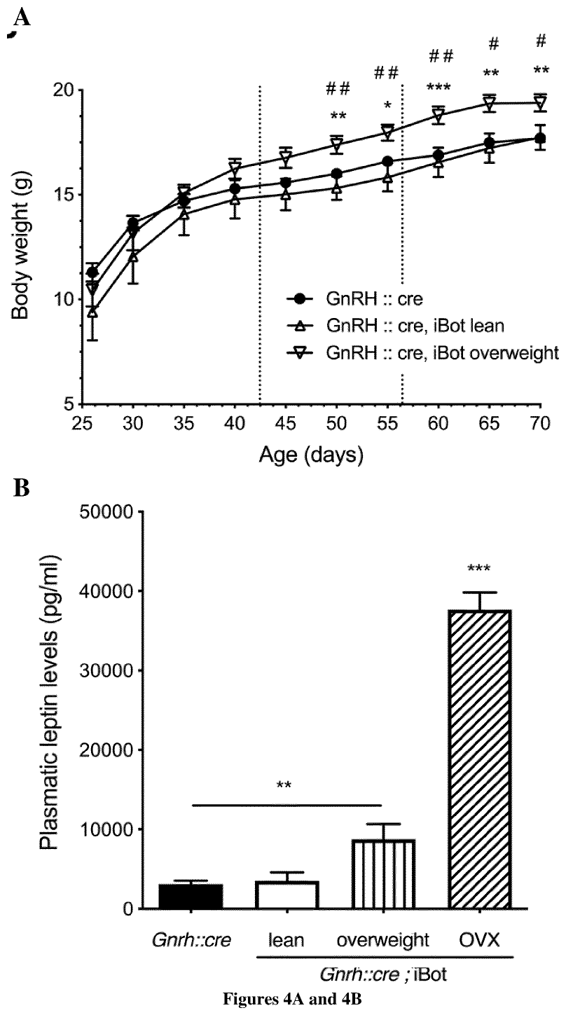

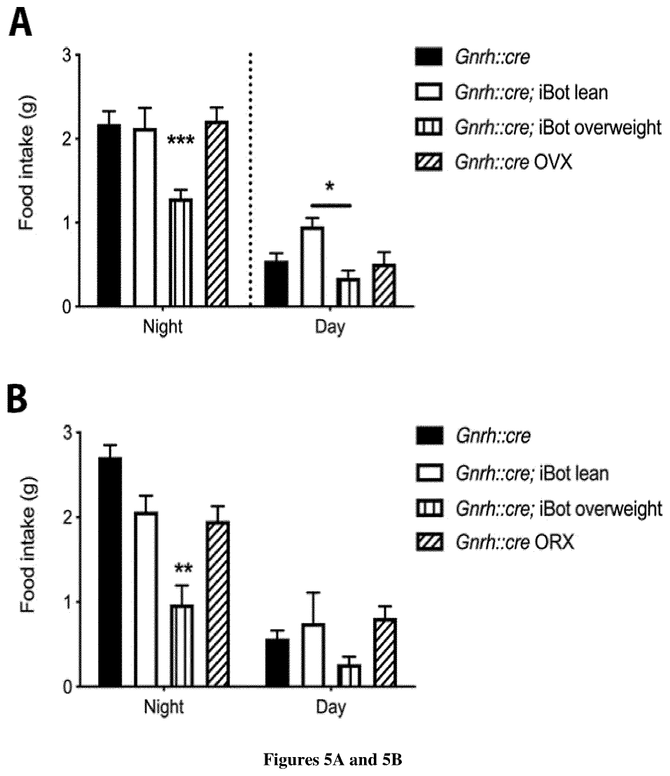

To further understand the implication of GnRH neurons and of GnRH in the development of the organism, activity-dependent exocytosis was selectively blocked in GnRH neurons by the expression of Clostridium botulinum neurotoxin serotype B (BoNT/B) (FIG. 1A). Without altering the migration and the distribution of GnRH neurons in the brain, BoNT/B expression in GnRH neurons inhibits the release of GnRH (FIG. 1B) and other co-released neuropeptides and thereby prevents GnRH from targeting its downstream targets both in the brain and in the pituitary. Interestingly, a subpopulation of female mutant mice expressing BoNT/B in GnRH neurons (Gnrh::cre;iBot overweight), in which the penetrance of the transgene was stronger, did not only fail to reach puberty onset (FIGS. 2A and 2B) and became hypogonadotropic hypogonadism (FIGS. 3A-3E), but also developed overweight and hyperleptinemia (FIGS. 4A and 4B). Unexpectedly, we found that the increase in body weight in mutant mice persisted in adulthood despite a significant decrease in food intake (FIGS. 5A and 5B), a phenomenon that is independent of the gonads. Together, these results point towards GnRH neurons establishing a link between sexual maturation and fertility, and metabolism.

On the whole, it becomes increasingly clear that GnRH neurons could constitute a link between fertility and metabolism, and that alteration in GnRH neuronal function could lead to fertility disorders and metabolic comorbidities. The pulsatile pattern of GnRH/LH release is well known to be critical for the normal functioning of the HPG-axis and fertility, but has recently been involved also in cognitive processes (see EP19305550.6). However, little is known about its importance in the regulation of food intake and energy homeostasis. Considering the fact that fertility can be restored in patients with hypogonadotropic hypogonadism by restoring the GnRH/LH pulsatile and that alterations in the pulsatile profile of LH release are observed in undernutrition/overnutrition, we further investigated whether restoring pulsatile GnRH/LH release could possibly have an impact on metabolic factors such as appetite.

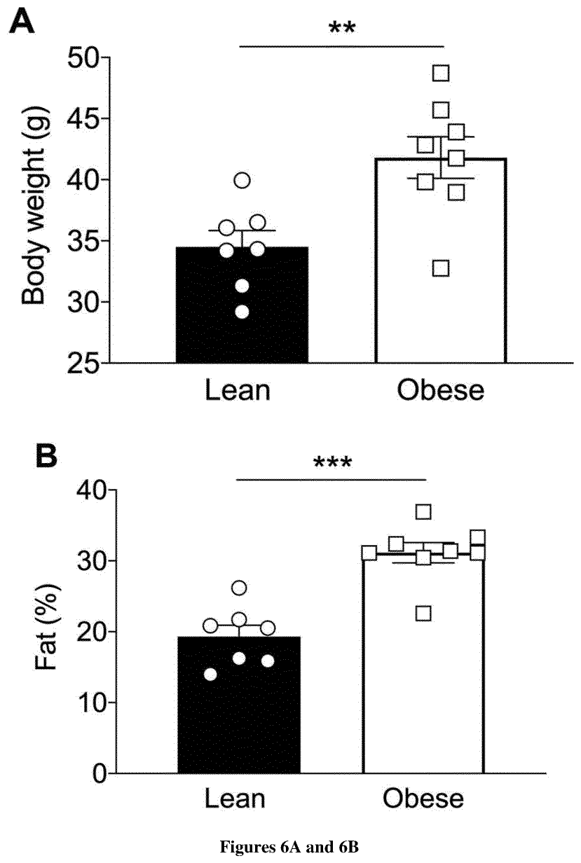

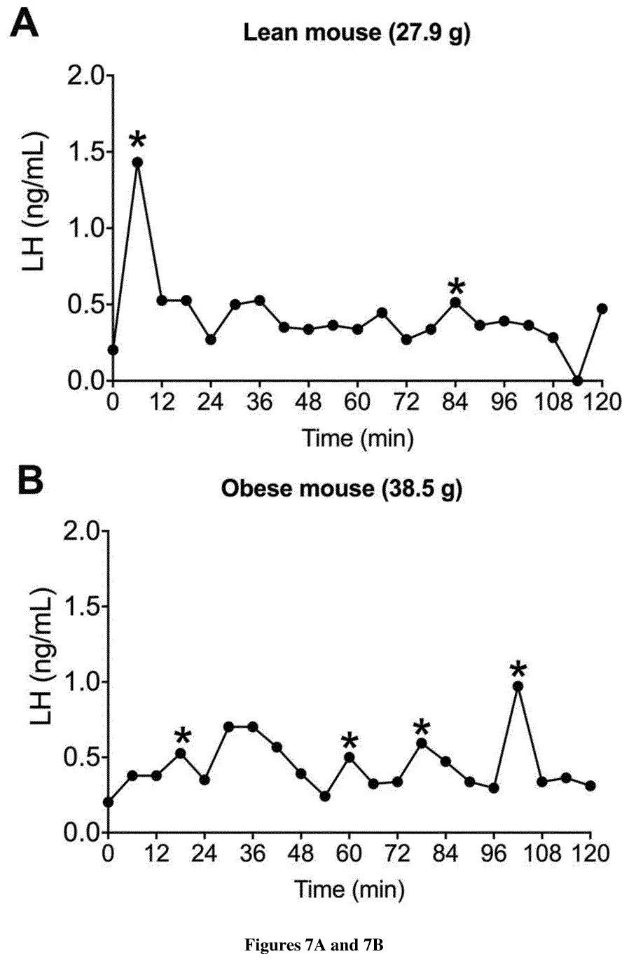

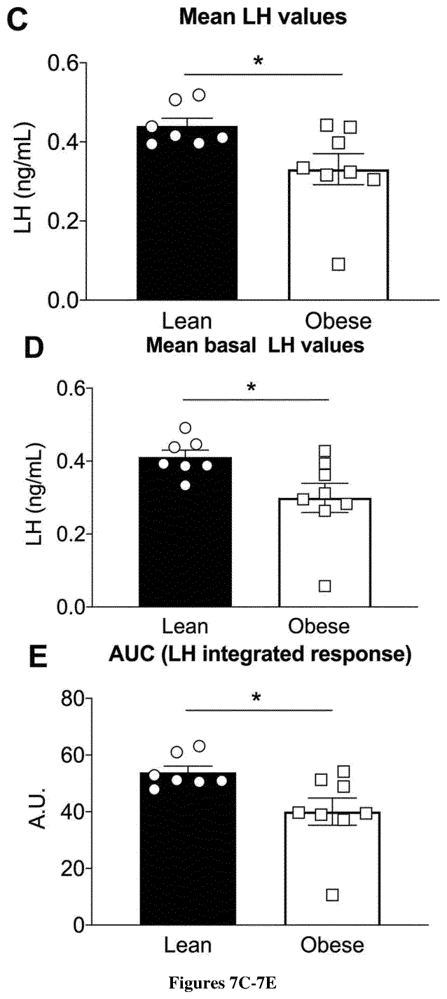

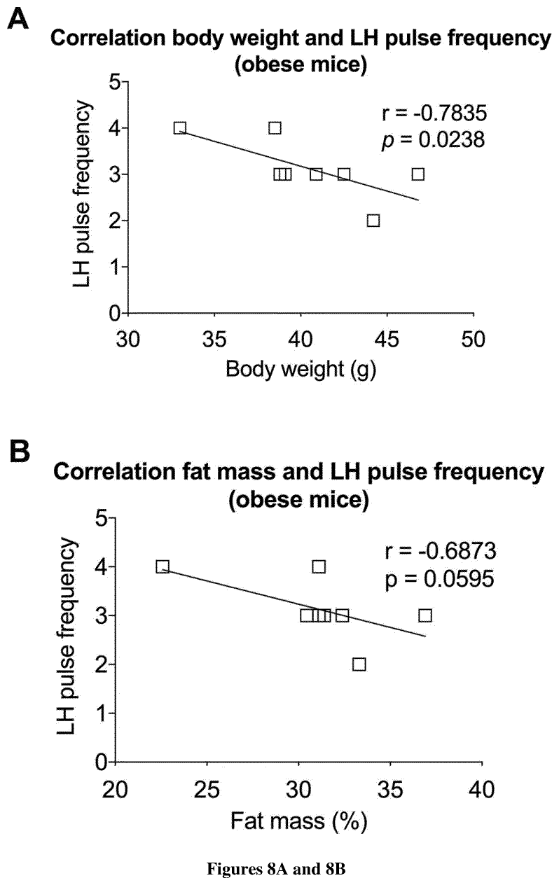

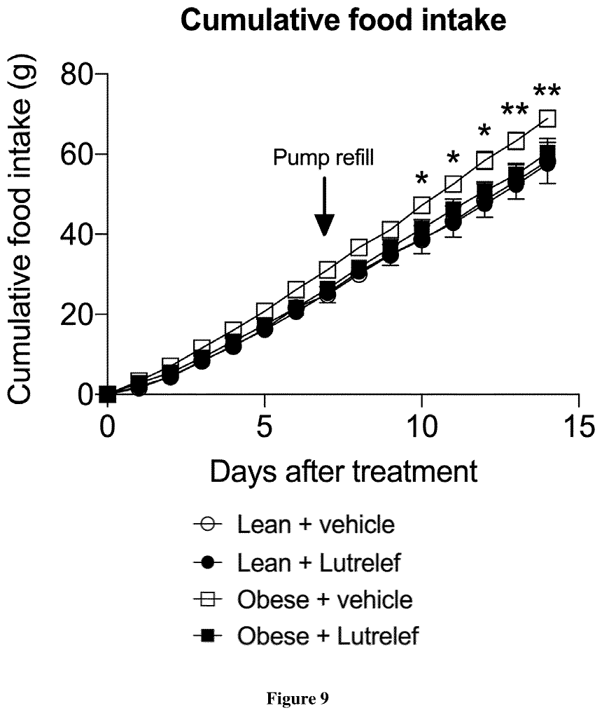

Obese mice and wildtype littermates (FIGS. 6A and 6B) were subjected to serial tail-tip blood sampling to determine the pattern of LH secretion. We observed a disruption of the pulsatile profile of LH release in obese mice, showing significantly reduced basal and average LH values (FIGS. 7A-7D) and LH integrated response as measured by the area under the curve (FIG. 7E). Moreover, correlation analyses showed that, in obese mice, the frequency of LH pulses is inversely correlated to body weight (FIG. 8A) and adiposity (FIG. 8B) demonstrating that the heavier the animals were getting the more the GnRH/LH pulsatile pattern was altered. To restore a control/lean pattern of GnRH/LH release in obese mice, we next implanted subcutaneous programmable mini-pumps that delivered either Lutrelef (0.25 μg of GnRH per pulse over 10 min given every 3 h) or vehicle to the mice (i.e., the pattern of secretion found in lean mice). Lutrelef is a native GnRH peptide that is clinically used to successfully treat hypogonadotropic infertility (23,24). The regimen was given for 14-days, during which body weight and food intake were followed daily. Pulsatile administration of a “lean pattern” of native GnRH peptide in obese mice normalised their cumulative food intake to levels comparable with lean mice, while it had no observable effect on the amount of food eaten by lean mice compared with their littermates that were infused with a vehicle solution (FIG. 9).

Here, we demonstrate that the pulsatile administration of a native GnRH peptide with a pattern found in lean mice reduces the amount of food consumed by obese mice to values comparable with their lean littermates. The pulsatile pattern of GnRH release does not only appear to be crucial for fertility, but also for metabolic fitness. Conversely, alterations in the pattern of GnRH release can impair energy homeostasis and lead to metabolic complications. Our results do not only highlight a previously unexpected role for GnRH in body homeostasis, but opens new therapeutic avenues in metabolic disorders, including hyperphagia, using an endogenous peptide already broadly used in the clinic for more than 35 years and that have proven to have no side effects.

### Example 2

### Definitions

The term “overweight” in females and “overlean” in males encompasses the term “sensitive” and the term “lean” encompasses the term “resistant”.

### Results:

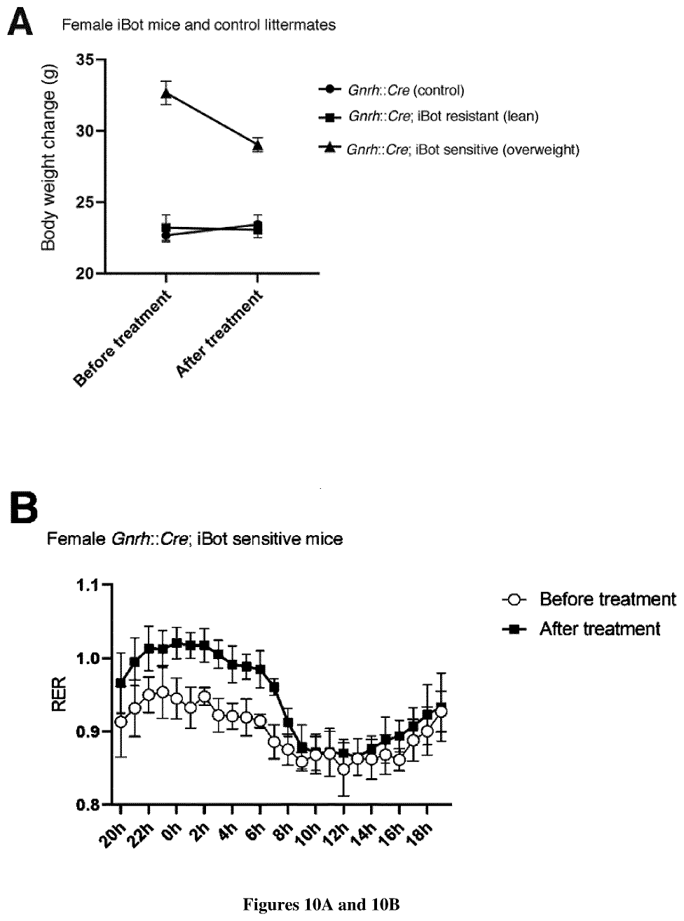

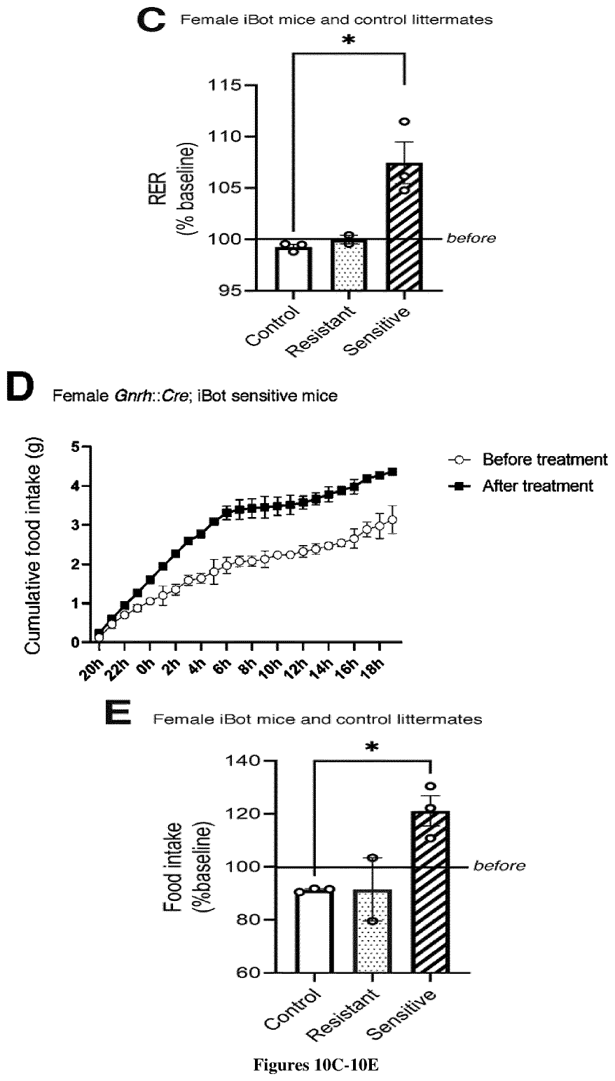

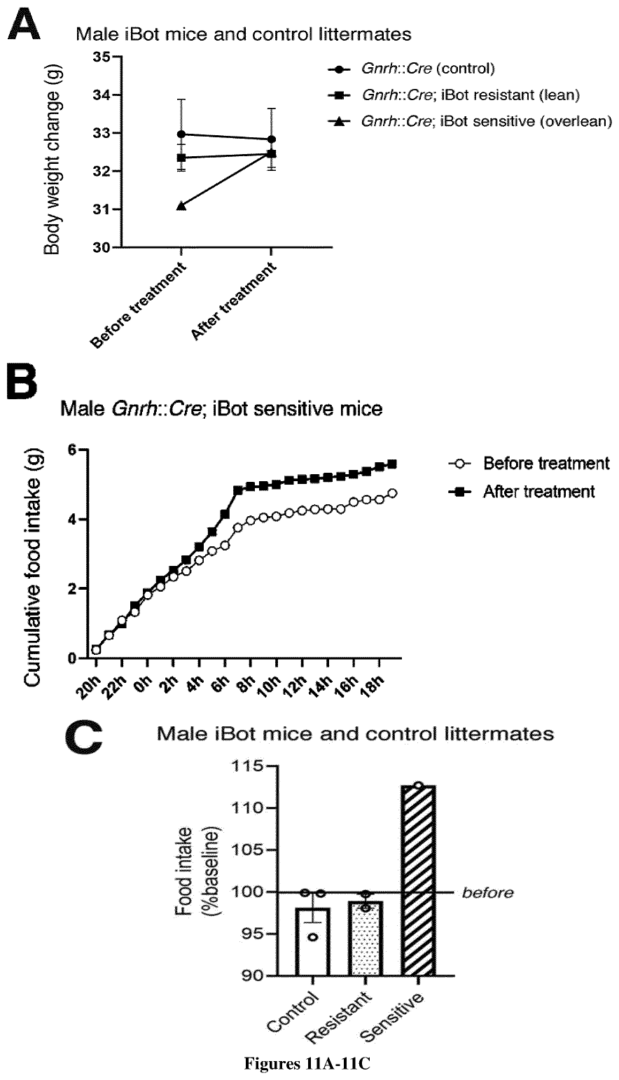

To determine whether reinstating pulsatile GnRH release in the Gnrh::Cre; iBot mice (FIG. 1A) sensitive to BoNT/B expression corrects their metabolic phenotype, we subcutaneously-implanted minipump delivering pulsatile Lutrelef during 15-day. Intriguingly, while female mice sensitive to the expression of BoNT/B in GnRH neurons showed overweight (FIG. 4A, FIG. 10A) despite decreased food intake (FIG. 5A), male mice sensitive to BoNT/B that ate less than their littermates resistant to the toxin (FIG. 5B) were found to be overlean (FIG. 11A), thus clearly showing a sexually dimorphic phenotype. Pulsatile administration of a “wildtype pattern” of native GnRH peptide in Gnrh::Cre; iBot sensitive mice normalised their phenotype both in females (FIG. 10) and in males (FIG. 11).

In Gnrh::Cre; iBot sensitive females, pulsatile Lutrelef delivery (one pulse every 2 h) decreased body weight (FIG. 10A), normalised the respiratory exchange ratio (RER: ratio of CO₂ produced by the body to the 02 consumed by the body; it gives an indication of the preferred use of certain substrates for energy homeostasis purposes: high RER indicates that the body consumes carbohydrates to operate its metabolism, while low RER indicates that the body mainly consumes lipids) (FIGS. 10B and 10C), and food intake (FIGS. 10D and 10E). In particular, RER data showed that pulsatile Lutrelef treatment restores the use of carbohydrates during the dark phase, i.e., during the feeding period, in Gnrh::Cre; iBot sensitive female mice (FIG. 10B). Minipump implantation resulted in no change in body weight, RER, and food intake in either Gnrh::Cre or resistant Gnrh::Cre; iBot mice. (FIGS. 10A, 10C and 10E).

In a Gnrh::Cre; iBot sensitive male, pulsatile Lutrelef delivery (one pulse every 3 h) induces a 15% increase in food intake (FIGS. 11B and 11C) and fully rescues bodyweight (FIG. 11A). Minipump implantation resulted in no change in body weight, RER, and food intake in either Gnrh::Cre or resistant Gnrh::Cre; iBot mice. (FIGS. 11A and 11C). Overall, these data strongly suggest that GnRH neuronal function and, in particular, pulsatile GnRH release is tightly involved in the control of food intake, weight balance and the proper functioning of energy metabolism.

### REFERENCES

Throughout this application, various references describe the state of the art to which this invention pertains. The disclosures of these references are hereby incorporated by reference into the present disclosure.
1. Plant T M, Terasawa E, Witchel S F. Puberty in non human primates and man. In: Plant T M, Zeleznik A J, eds. Knobil and Neill's physiology of reproduction. San Diego: Academic Press; 2015:1487-1536.
2. Belchetz P E, Plant T M, Nakai Y, Keogh E J, Knobil E. Hypophysial responses to continuous and intermittent delivery of hypopthalamic gonadotropin-releasing hormone. Science. 1978; 202(4368):631-633.
3. Jayasena C N, Abbara A, Veldhuis J D, Comninos A N, Ratnasabapathy R, De Silva A, Nijher G M, Ganiyu-Dada Z, Mehta A, Todd C, Ghatei M A, Bloom S R, Dhillo W S. Increasing L H pulsatility in women with hypothalamic amenorrhea using intravenous infusion of Kisspeptin-54. J Clin Endocrinol Metab. 2014; 99(6):E953-961.
4. Germain N, Fauconnier A, Klein J P, Wargny A, Khalfallah Y, Papastathi-Boureau C, Estour B, Galusca B. Pulsatile gonadotropin-releasing hormone therapy in persistent amenorrheic weight-recovered anorexia nervosa patients. Fertil Steril. 2017; 107(2):502-509.
5. Boehm U, Bouloux P M, Dattani M T, de Roux N, Dode C, Dunkel L, Dwyer A A, Giacobini P, Hardelin J P, Juul A, Maghnie M, Pitteloud N, Prevot V, Raivio T, Tena-Sempere M, Quinton R, Young J. Expert consensus document: European Consensus Statement on congenital hypogonadotropic hypogonadism—pathogenesis, diagnosis and treatment. Nat Rev Endocrinol. 2015; 11(9):547-564.
6. Navarro V M. Metabolic regulation of kisspeptin—the link between energy balance and reproduction. Nat Rev Endocrinol. 2020.
7. Herde M K, Geist K, Campbell R E, Herbison A E. Gonadotropin-releasing hormone neurons extend complex highly branched dendritic trees outside the blood-brain barrier. Endocrinology. 2011; 152(10):3832-3841.
8. Roland A V, Moenter S M. Glucosensing by GnRH neurons: inhibition by androgens and involvement of AMP-activated protein kinase. Mol Endocrinol. 2011; 25(5):847-858.
9. Rosenfield R L, Bordini B. Evidence that obesity and androgens have independent and opposing effects on gonadotropin production from puberty to maturity. Brain Res. 2010; 1364:186-197.
10. Rosenbaum M, Leibel R L. The role of leptin in human physiology. N Engl J Med. 1999; 341(12):913-915.
11. Welt C K, Chan J L, Bullen J, Murphy R, Smith P, DePaoli A M, Karalis A, Mantzoros C S. Recombinant human leptin in women with hypothalamic amenorrhea. N Engl J Med. 2004; 351(10):987-997.
12. Watanobe H, Habu S. Leptin regulates growth hormone-releasing factor, somatostatin, and alpha-melanocyte-stimulating hormone but not neuropeptide Y release in rat hypothalamus in vivo: relation with growth hormone secretion. J Neurosci. 2002; 22(14): 6265-6271.
13. Parent A S, Teilmann G, Juul A, Skakkebaek N E, Toppari J, Bourguignon J P. The timing of normal puberty and the age limits of sexual precocity: variations around the world, secular trends, and changes after migration. Endocr Rev. 2003; 24(5):668-693.
14. Howard S R, Dunkel L. Delayed Puberty-Phenotypic Diversity, Molecular Genetic Mechanisms, and Recent Discoveries. Endocr Rev. 2019; 40(5):1285-1317.
15. Abraham S F, Pettigrew B, Boyd C, Russell J. Predictors of functional and exercise amenorrhea among eating and exercise disordered patients. Hum Reprod. 2006; 21(1):257-261.
16. Mason H D, Key A, Allan R, Lask B. Pelvic ultrasonography in anorexia nervosa: what the clinician should ask the radiologist and how to use the information provided. Eur Eat Disord Rev. 2007; 15(1):35-41.
17. Zumoff B, Strain G W, Miller L K, Rosner W, Senie R, Seres D S, Rosenfeld R S. Plasma free and non-sex-hormone-binding-globulin-bound testosterone are decreased in obese men in proportion to their degree of obesity. J Clin Endocrinol Metab. 1990; 71(4):929-931.
18. Farooqi I S. Leptin and the onset of puberty: insights from rodent and human genetics. Semin Reprod Med. 2002; 20(2):139-144.
19. Apter D. The role of leptin in female adolescence. Ann N Y Acad Sci. 2003; 997:64-76.
20. Tsai E C, Boyko E J, Leonetti D L, Fujimoto W Y. Low serum testosterone level as a predictor of increased visceral fat in Japanese-American men. Int J Obes Relat Metab Disord. 2000; 24(4):485-491.
21. Saylor P J, Smith M R. Metabolic complications of androgen deprivation therapy for prostate cancer. J Urol. 2013; 189(1 Suppl):534-42; discussion S43-34.
22. Vanacker C, Trova S, Shruti S, Casoni F, Messina A, Croizier S, Malone S, Ternier G, Hanchate N K, Bouret S G, Ciofi P, Giacobini P, Prevot V. Neuropilin-1 expression in GnRH neurons regulates prepubertal weight gain and sexual attraction. Embo J. 2020: e104633.
23. Hoffman A R, Crowley W F, Jr. Induction of puberty in men by long-term pulsatile administration of low-dose gonadotropin-releasing hormone. N Engl J Med. 1982; 307(20): 1237-1241.
24. Hurley D M, Brian R, Outch K, Stockdale J, Fry A, Hackman C, Clarke I, Burger H G. Induction of ovulation and fertility in amenorrheic women by pulsatile low-dose gonadotropin-releasing hormone. N Engl J Med. 1984; 310(17):1069-1074.

1. A method of treating a food intake related disorder in a patient in need thereof, comprising administering to the patient a therapeutically effective amount of gonadotropin-releasing hormone (GnRH), wherein said step of administering is conducted by pulsatile administration.

2. A method of treating a food intake related disorder in a patient in need thereof comprising administering to the patient a therapeutically effective dose of one or more of miR-200, a compound mimicking miR-200, a miR-155, and a compound mimicking miR-155.

3. The method according to claim 1, wherein said food intake related disorder is obesity, an obesity related diseases, being overweight, overeating, anorexia cachexia syndrome (ACS), anorexia nervosa, being underweight or under eating.

4. The method according to claim 3, wherein said food intake related disorder is obesity.

5. The method according to claim 3, wherein said food intake related disorder is anorexia cachexia syndrome (ACS) or anorexia nervosa.

6. The method according to claim 1, wherein said patient is a man.

7. The method according to claim 6, wherein said pulsatile administration corresponds to an administration of 25 ng/kg of GnRH every 120 minutes.

8. The method according to claim 1, wherein said patient is a woman.

9. The method according to claim 8, wherein said pulsatile administration corresponds to an administration of 75 ng/kg of GnRH every 90 minutes.

10. The method according to claim 1, wherein said GnRH is gonadorelin.

11. The method according to claim 2, wherein said food intake related disorder is obesity, an obesity related disease, being overweight, overeating, anorexia cachexia syndrome (ACS), anorexia nervosa, being underweight or under eating.

12. The method according to claim 11, wherein said food intake related disorder is obesity.

13. The method according to claim 11, wherein said food intake related disorder is anorexia cachexia syndrome (ACS) or anorexia nervosa.

14. The method according to claim 2, wherein said patient is a man.

15. The method according to claim 2, wherein said patient is a woman.
